(19) United States
(12) Patent Application Publication
(10) Pub. No.: US 2021/0222941 A1
(43) Pub. Date: Jul. 22, 2021

(54) MODULAR TEMPERATURE CONTROL DEVICE AND METHOD

(71) Applicant: L&G Barbeque Grills LLC

(72) Inventors: Leonidas Carayannopoulos, Woburn, MA (US); George Tsitsopoulos, Woburn, MA (US); Zack Tucci, Woburn, MA (US); Stamatis Carayannopoulos, Woburn, MA (US)

(21) Appl. No.: 16/748,245

(22) Filed: Jan. 21, 2020

(51) Int. Cl. F25D 3/00; A61J 1/16; B65D 81/18; A61F 7/10

(52) U.S. Cl. CPC F25D 3/00; A61J 1/165; A61F 2007/0008; A61F 7/10; B65D 81/18

(57) ABSTRACT

Described embodiments provide systems and methods for managing the temperature of an item that includes a first flap portion and a second flap portion defining between them a first pouch containing substance; a third flap portion and the second flap portion defining between them a second pouch containing substance; a first portion of a fastener attached to the front side of the first flap portion; a first complementary portion of the fastener attached to the obverse side of the second flap portion; a second complementary portion of the fastener attached to the obverse side of the third flap portion.

### FIELD OF THE DISCLOSURE

The present application generally relates to a modular temperature control device for maintaining the temperature of an item.

### BACKGROUND

Temperature control of perishable items is important for preservation, but existing designs are bulky and impractical. For example, not all perishable items fit inside a rigid wall container. Rigid wall containers are impractical for moving to the perishable items. Similarly, ice baths are impractical for targeting temperature regulation to a particular location of the item, but rather require temperature regulation of the environment inside the ice bath. It is also difficult to predict how much energy for temperature regulation the item needs and where on the item the temperature regulation will need to be applied. Also, rigid temperature control devices are difficult to transport to the location of where the item is located. Likewise, temperature control devices of a preset size are difficult to adjust to different items.

### SUMMARY

This Summary is provided to introduce a selection of concepts in a simplified form that are further described below in the Detailed Description. This Summary is not intended to identify key features or essential features, nor is it intended to limit the scope of the claims included herewith.

This disclosure describes a method and system for temperature control of an item such as, but not limited to, meat and food. An aspect of this disclosure provides an apparatus for temperature control of a variety of items. The apparatus includes a temperature control device with pouches, flaps, and fasteners. The pouches may include a temperature control substance. A temperature control device can attach to other temperature control devices to form a larger temperature control device through the use of fasteners, temperature control device. The temperature control devices may have different sizes and shapes. To control the temperature of an item, a temperature control device is assembled, wrapped around the item and secured. The temperature control devices are flexible, and mold to the shape of the item to create a targeted temperature controlled zone.

In one aspect, a flexible system for controlling the temperature of the item includes a first flap portion and a second flap portion defining between them a first pouch containing a substance. A third flap portion and the second flap portion define between them a second pouch containing a substance. A first portion of a hook and loop fastener attaches to the front side of the first flap portion. A first complementary portion of the hook and loop fastener attaches to the obverse side of the second flap portion. A second complementary portion of the hook and loop fastener attaches to the obverse side of the third flap portion.

In some implementations, a second portion of the hook and loop fastener attaches to the front side of the second flap portion. In certain implementations, the first portion of the hook and loop fastener has a first surface area equal to a second surface area of the second portion of the hook and loop fastener. In certain implementations, the first portion of the hook and loop fastener is a square shape.

In some implementations, the first complementary portion of the hook and loop fastener has a third surface area equal to a fourth surface area of the second complementary portion of the hook and loop fastener. In some implementations, the first complementary portion of the hook and loop fastener is a square shape. In some implementations, the temperature control substance is a gel. In some implementations, the temperature control substance is propylene glycol and water. In some implementations, the first pouch has a different amount of the temperature control substance from the second pouch. In some implementations, a surface area of the first, second, and third flap and a surface area of the first pouch and the second pouch are equal. In some implementations, the first complementary portion of the hook and loop fastener comprises a strip of material. In some implementations, the first portion of the hook and loop fastener comprises a strip of material. In some implementations, the second complementary portion of the hook and loop fastener comprises a strip of material. In certain implementations, the second portion of the hook and loop fastener is a strip of material.

In some implementations, the first complementary portion of the hook and loop fastener a plurality of shapes attaches to the obverse side of the first flap portion.

In another aspect, a method for applying targeted temperature control includes providing a first temperature control apparatus having a first flap portion and a second flap portion defining between them a first pouch containing substance. A third flap portion and the second flap portion defining between them a second pouch containing substance. A first portion of a hook and loop fastener attaches to the front side of the first flap portion. A first complementary portion of the hook and loop fastener attaches to the obverse side of the second flap portion. A second complementary portion of the hook and loop fastener attaches to the obverse side of the third flap portion. The first apparatus wraps around an item.

In some implementations, the method includes securing the first apparatus to the item by affixing the first portion of the hook and loop fastener to the first complementary portion of the hook and loop fastener. In some implementations, the method includes securing the first apparatus to the item by affixing the first portion of the hook and loop fastener to the second complementary portion of the hook and loop fastener. In some implementations, the method includes providing a second temperature control apparatus comprising a first flap portion and a second flap portion defining between them a first pouch containing substance, a third flap portion and the second flap portion defining between them a second pouch containing substance, a first portion of a hook and loop fastener attached to the front side of the first flap portion, a first complementary portion of the hook and loop fastener attached to the obverse side of the second flap portion, and a second complementary portion of the hook and loop fastener attached to the obverse side of the third flap portion. In certain implementations, the method includes attaching the first portion of the second temperature control apparatus to the second complementary portion of the first temperature control apparatus to form a combined apparatus, wrapping the combined apparatus around an item; and securing the combined apparatus to the item by affixing the first portion of the first temperature control apparatus to the second complementary portion of the first temperature control apparatus. In certain implementations, the method includes coupling the first complementary portion of the hook and loop fastener of the first temperature control apparatus and the first complementary portion of the hook and loop fastener of the second temperature control apparatus to a cover comprising a first and a second complementary portion of a hook and loop fastener.

### BRIEF DESCRIPTION OF THE DRAWINGS

Objects, aspects, features, and advantages of embodiments disclosed herein will become more fully apparent from the following detailed description, the appended claims, and the accompanying drawing figures in which like reference numerals identify similar or identical elements. Reference numerals that are introduced in the specification in association with a drawing figure may be repeated in one or more subsequent figures without additional description in the specification in order to provide context for other features, and not every element may be labeled in every figure. The drawing figures are not necessarily to scale, emphasis instead being placed upon illustrating embodiments, principles and concepts. The drawings are not intended to limit the scope of the claims included herewith.

FIG. 1A is a diagram depicting of a front side of a temperature control device;

FIG. 1B is a diagram depicting of an obverse side of a temperature control device;

FIG. 1C is a diagram depicting of a cross section view of a temperature control device;

FIG. 2A is a diagram depicting two temperature control devices just prior to being assembled;

FIG. 2B is a diagram depicting two temperature control devices after assembly;

FIG. 3 is a diagram depicting the temperature control device in a bag;

FIG. 4 is a diagram depicting the temperature control device around an animal;

FIG. 5 is a diagram depicting the temperature control device around a fish;

FIG. 6 is a diagram depicting the temperature control device around a leg;

FIG. 7 is a diagram depicting the temperature control device around a head;

FIG. 8 is a diagram depicting the temperature control device around medication;

FIG. 9 is a diagram depicting various embodiments of the temperature control device;

FIG. 10 is a diagram depicting the temperature control device secured around an item;

### DETAILED DESCRIPTION

For purposes of reading the description of the various embodiments below, the following descriptions of the sections of the specification and their respective contents may be helpful:

The present disclosure describes systems and methods for a temperature control device. Items have desired temperatures for preservation. Controlling the temperature of items is one way to for keep items preserved and at their appropriate temperature. Items might have a variety of shapes, so a rigid temperature control design might not be able to control the temperature of every part of the item, whereas a flexible temperature control design could adapt to each section of the item. The modular temperature control device presented is attached to the items to provide targeted temperature control.

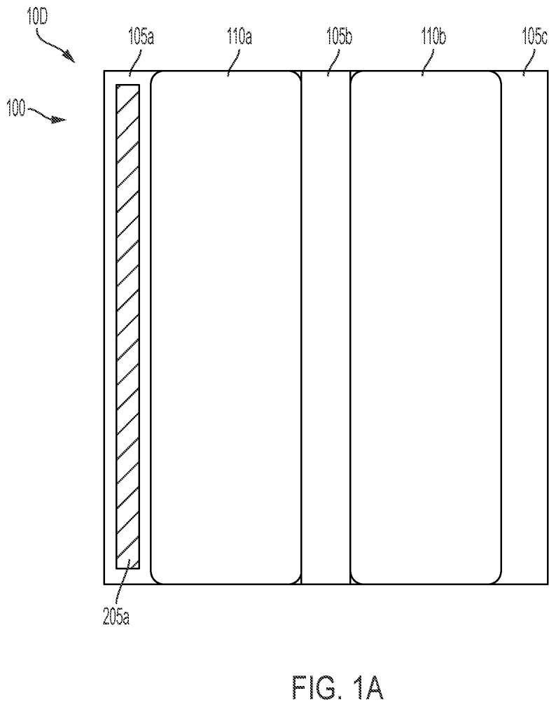

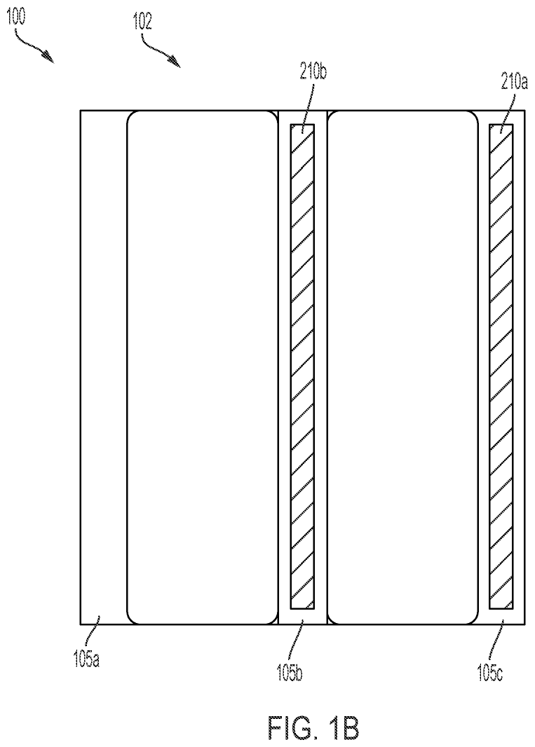

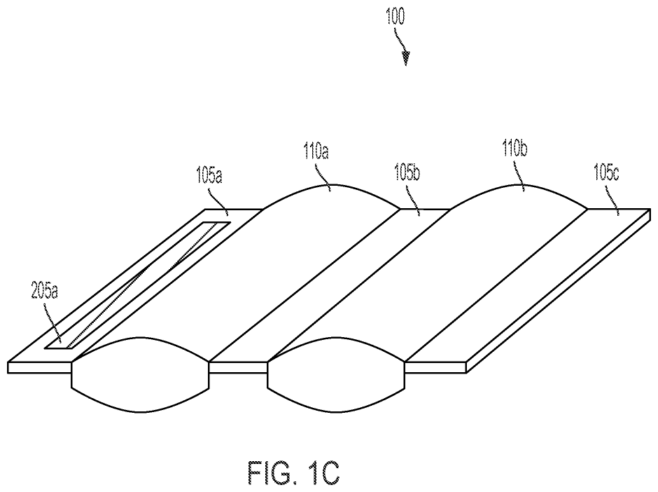

Referring now to FIG. 1A, and in brief overview, a front side of a temperature control device 100 is shown. The temperature control device 100 comprises pouches 110a-110b (generally referred to as a pouch 110), and flaps 105a-105c (generally referred to as a flap 105). As shown in FIG. 1A, pouch 110a is coupled to a flap 105a and a flap 105b and pouch 110b is coupled to the flap 105b and a flap 105c. As shown in FIG. 1A, flap 105a includes a fastener 205a (generally referred to as fastener 205). The fastener 205a can be a portion of a hook and loop fastener.

Still referring to FIG. 1A, and in more detail, the temperature control device 100 includes pouches 110a, 110b coupled to flaps 105a, 105b, 105c. In some implementation, pouches 110 may be affixed to flaps 105 using any suitable adhesive. In other embodiments, pouch 110 and flaps 105 may be formed as a unitary structure by combining two sheets of material, such as high-density polyethylene, and heat-welding the seam between the pouch 110 and the flap 105. In some implementations, the surface area of the pouch 110 is equal to the surface area of the flap 105. In some implementations, the surface area of the pouch 110 is greater than the surface area of the flap 105. For example, the surface area of the pouch 110 may be up to double the surface area of the flaps 105. In still other embodiments, the surface area of the pouch 110 may be up to triple the surface area of the flaps 105. In still other embodiments, the flaps 105 may have different surface areas from each other. In these embodiments, the ratio of the surface area of the pouch 110 to the flap 105 may be calculated with respect to either of the flaps 105.

Still referring to FIG. 1A, the temperature control device 100 includes one or more fastening devices or securing mechanisms. As shown in FIG. 1A, flap 105a includes a portion of a fastener 205a (generally referred to as fastener 205). In some embodiments, fastener 205 is a hook-and-loop fastener, such as VELCRO. In other embodiments, fastener 205 may be a single-use adhesive or a multiple-use adhesive. In still other embodiments, fastener 205 may be a zipper, button, snap or hook-and-eye fastener. In some implementations, the surface area of the fastener 205 is equal to the surface area of the flap 105. Although the fastener 205 in FIG. 1A is shown as a continuous strip, in other embodiments the fastener 205 may be discrete elements disposed on the flap 105, such as buttons, snaps, or VELCRO dots. In some embodiments, the fastener 205 is affixed to flap 105 using adhesive. In other embodiments, the fastener 205 is fabricated as a unitary portion of the flap 105. In some implementations, the fastener 205 is fabricated from plastic, metal, or cloth and may be removable from flap 105 or permanent. Referring now FIG. 1B, and in brief overview, the obverse side of the temperature control device 100. As shown in FIG. 1B, the obverse side of flaps 105b, 105c include fastener complementary portions 210a-210b of the fastener 205. As shown in FIG. 1B, flap 105c includes fastener complementary portion 210a, and flap 105b includes fastener complementary portion 210b. In some embodiments, fastener complementary portion 210 is a hook-and-loop fastener, such as VELCRO. In other embodiments, fastener complementary portion 210 may be a single-use adhesive or a multiple-use adhesive. In still other embodiments, fastener complementary portion 210 may be a zipper, button, snap or hook-and-eye fastener. In some implementations, the surface area of the fastener complementary portion 210 is equal to the surface area of the flap 105. Although the fastener complementary portion 210 in FIG. 1B is shown as a continuous strip, in other embodiments the fastener complementary portion 210 may be discrete elements disposed on the flap 105, such as buttons, snaps, or VELCRO dots. In some embodiments, the fastener complementary portion 210 is affixed to flap 105 using adhesive. In other embodiments, the fastener complementary portion 210 is fabricated as a unitary portion of the flap 105. In some embodiments, fastener complementary portions 210a, 210b are complementary portions for different types of fasteners 205. In still other embodiments, the temperature control device 100 may include a fastener complementary portion 210 on only one of the flaps 105. In some implementations, the fastener complementary portion 210 may be fabricated from plastic, metal, or cloth and may be removable from flap 105 or permanent.

Referring now to FIG. 1C, a cross-sectional view of the temperature control device 100 is shown. The pouch 110 includes a substance for maintaining the temperature of an item. In some implementations, the substance is a gel. In other implementations, the substance is a liquid. In still other embodiments, the substance may be solid pellets. In some specific implementations, the substance may by propylene glycol, water, or a combination of the two. In some of these embodiments, the substance is a mixture of 90% propylene glycol and 10% water. In other embodiments, the substance is 80% propylene glycol and 20% water. In still other embodiments, the substance is 70% propylene glycol and 30% water. In further embodiments, the substance is 60% propylene glycol and 40% water. In still further embodiments, the substance is 50% propylene glycol and 50% water. In some other embodiments, the substance is 40% propylene glycol and 60% water. In other embodiments, the substance is 30% propylene glycol and 70% water. In still other embodiments, the substance is 20% propylene glycol and 80% water. In still further embodiments, the substance is 10% propylene glycol and 90% water. In some implementations, pouch 110a contains a different substance or a different amount of a substance than pouch 110b.

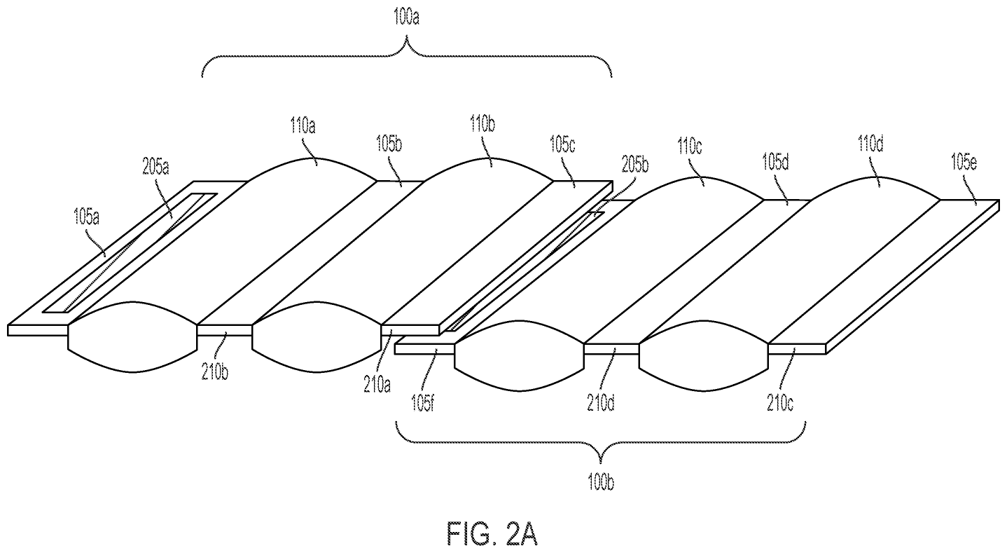

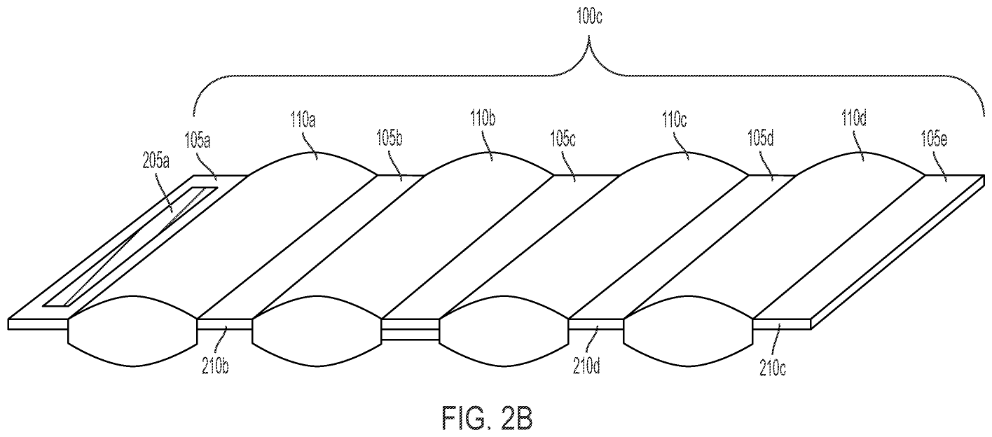

Referring now to FIG. 2A, depicted is a diagram of a temperature control device 100a just prior to attachment with temperature control device 100b. The temperature control device 100a is aligned with temperature control device 100b such that fastener complementary portion 210a on the obverse side of flap 105c of temperature control device 100a aligns with the fastener 205b on the front side of flap 105f of temperature control device 100b. FIG. 2B shows the result of attaching the temperature control device 100a to temperature control device 100b by mating the fastener complementary portion 210a on the obverse side of flap 105c of temperature control device 100a with the fastener 205b on the front side of flap 105f of temperature control device 100b. In certain embodiments, the fastener complementary portion 210a is heat-welded to the fastener 205b. As shown in FIG. 2B, resultant temperature control device includes four pouches 110a-110d for controlling temperature. Fasteners 205a of temperature control device 100a, fastener complementary portion 210c of temperature control device 100b, and fastener complementary portion 210d of temperature control device 100b are available for engagement with further temperature control devices 100. Although FIGS. 2A-2B depict the overlap of flap 105 of temperature control device 100a with flap 105f of temperature control device 100b to be substantially co-extensive, any overlap of the two flaps will suffice provided that the fastener components can mate. In some embodiments, flap 105c overlaps 90% of flap 105f. In some embodiments, flap 105c overlaps 90% of flap 105f. In other embodiments, flap 105c overlaps 80% of flap 105f. In still other embodiments, flap 105c overlaps 70% of flap 105f. In further embodiments, flap 105c overlaps 60% of flap 105f. In still further embodiments, flap 105c overlaps 50% of flap 105f. In still other embodiments, flap 105c overlaps 40% of flap 105f. In some other embodiments, flap 105c overlaps 30% of flap 105f. In some further embodiments, flap 105c overlaps 20% of flap 105f. In other embodiments, flap 105c overlaps 10% of flap 105f. When discussing the overlap of flaps, the percentage overlap may refer to overlap in either of the two axes of freedom.

Still referring to FIG. 2B, the formed temperature control device 100c can create varying temperature control zones by attaching a fastener 205a to a fastener complementary portion 210c or, alternatively, to fastener complementary portion 210b. In some implementations, the portion of 205a engages with the fastener complementary portion 210c to change the shape of the temperature control device 100c. By adjusting the shape of the temperature control device 100c, the temperature control device 100c can be placed at a variety of locations. The modularity of the temperature control device allows for selectable device placement. In some implementations, the temperature control device 100 folds for placement in a freezer or in an oven, to prepare the device.

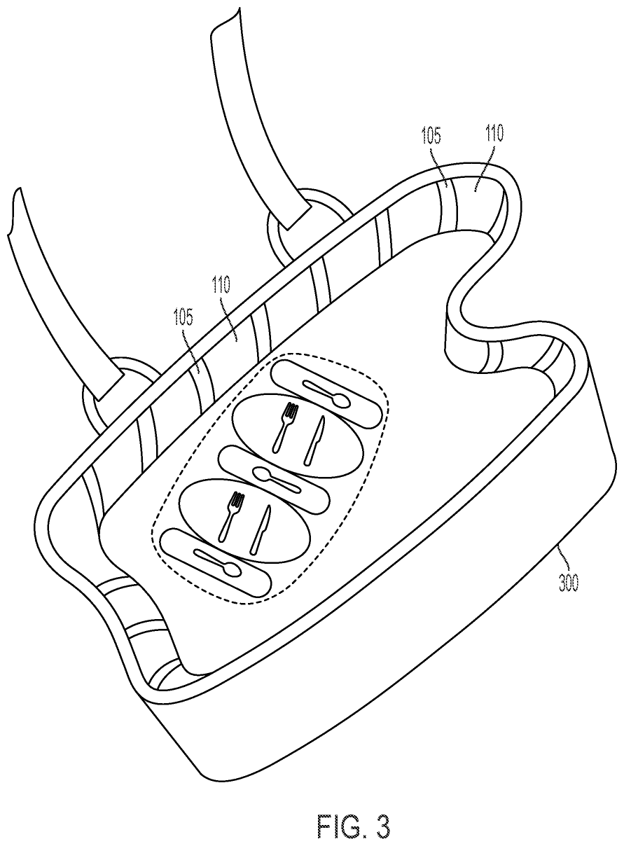

Referring now to FIG. 3, the temperature control device 100 is shown fixed to a bag 300. In some implementations, the bag 300 is a tote bag or any container capable of storing an item. In some implementations, the temperature control device 100 is used to cool the interior of bag 300 to keep items in the bag cool. In some implementations, the temperature control device 100 creates an unregulated temperature control zone in a container that creates a closed environment from the ambient environment. In some implementations, the temperature control device 100 is disposed around the inner perimeter of the tote bag to create a temperature control zone to store any type of perishables, such as fruits, vegetables, medication such as insulin, beverages, cold sandwiches, and ice cream. In alternative embodiments, the temperature control device 100 may be heated to keep items in the bag warm, such as when the item is a cooked piece of meat.

The bag 300 may be permanent or disposable. The temperature control device 100 can surround the item, as shown in FIG. 3, to provide 360 degrees (“fully surrounded”) of temperature management. In other embodiments, the temperature control device 100 may be fixed in the bag for only a portion of the perimeter. In these embodiments, temperature control may be provided for 270 degrees (“partially surrounded”), 180 degrees (“half surrounded”), or 90 degrees (“quarter surrounded”). In some implementations, the temperature control device 100 attaches to the bag 300. The bag 300 may include a fastener 205 capable of mating with the fastener complementary portion 210 provided by the temperature control device 100. The bag 300 may include a fastener complementary portion 210 capable of mating with the fastener 205 provided by the temperature control device 100. In these embodiments, the temperature control device 100 is attached to the internal side of the bag 300. In an alternate implementation, the bag may provide a pocket for receiving the temperature control device 100. In further embodiments, the temperature control device 100 may be attached to the outside of the bag 300. The fastener 205 or fastener complementary portion 210 of the temperature control device 100 engages with the fastener 205 or fastener complementary portion 210 of the bag 300. The temperature control device 100 provides temperature control for the bag 300 or the item inside the bag. In some implementations, the location of temperature control device 100 may be adjusted within the bag 300. Adjusting a temperature control device 100 location allows for the temperature management to be directed. Stated in another way, by placing the temperature control device 100 at different locations, different zones are created, allowing for optimized cooling or heating for the item. In some implementations, the location of the temperature control device 100 varies with the degree of proximity to the item.

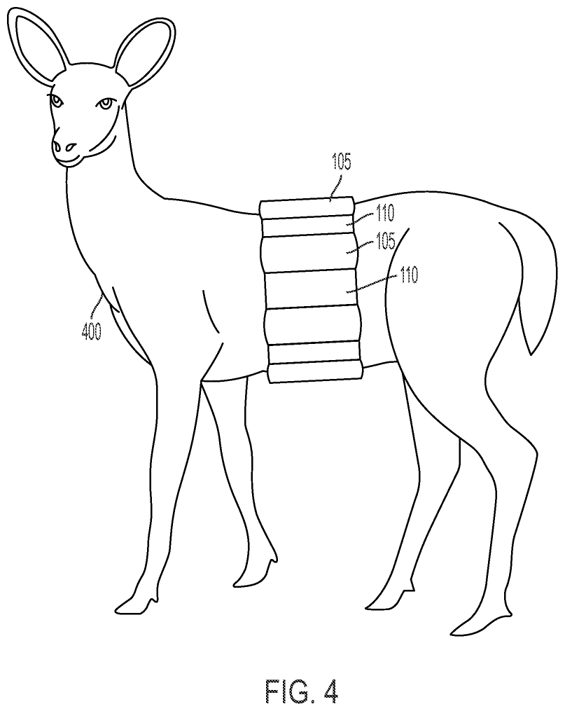

FIG. 4, depicts the temperature control device 100 disposed around an animal 400. In some implementations, the animal 400 is a deer, bear, or other carcass. The temperature control device is looped around the animal 400by engaging a fastener 205 or fastener complementary portion 210 of the front side of the temperature control device 100 to a fastener 205 or fastener complementary portion 210 of the obverse side of the temperature control device 100. The temperature control device 100 controls the temperature of large meats such as pigs, lambs, deer, quail that were recently shot while hunting. The temperature control device 100 may be disposed anywhere on the animal 400 and, in some embodiments, multiple temperature control devices 100 may be used to target temperate control to several portions of the animal. The temperature control device 100 adjusts to fit virtually any size animal. The temperature control device 100 is secured to the animal 400 by engaging a fastener 205 or a fastener complementary portion 210 of the temperature control device 100 to a fastener 205 or fastener complementary portion 210 of the temperature control device or animal 400 to dispose the temperature control device 100 in a continuous, circular, or square shape.

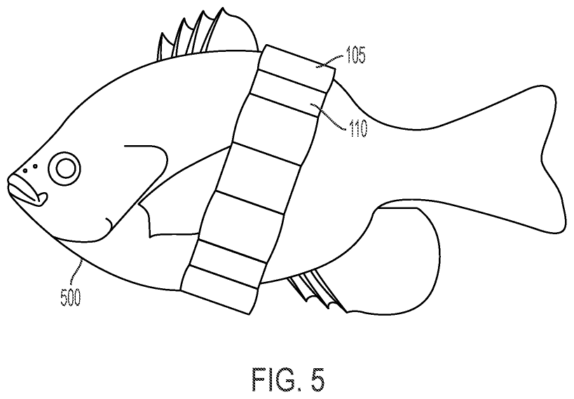

Referring to FIG. 5, depicted is the temperature control device 100 disposed around a fish 500. In some implementations, the fish 500 is a bass, trout, shark, or any other sea creature. In some implementations, the temperature control device 100 controls the temperature of fish 500 that are freshly caught. The temperature control device 100 may be disposed anywhere on the fish 500. In some implementations, the temperature control device 100 is wrapped around the fish 500. The temperature control device 100 is secured to the fish 500 by engaging a fastener 205 or a fastener complementary portion 210 of the temperature control device 100 to a fastener 205 or fastener complementary portion 210 of the temperature control device 100 or fish 500 to dispose the temperature control device 100 in a continuous, circular, or square shape. In some implementations, by connecting the temperature control device 100 along the longitude, it will wrap a fish 500 from head to tail.

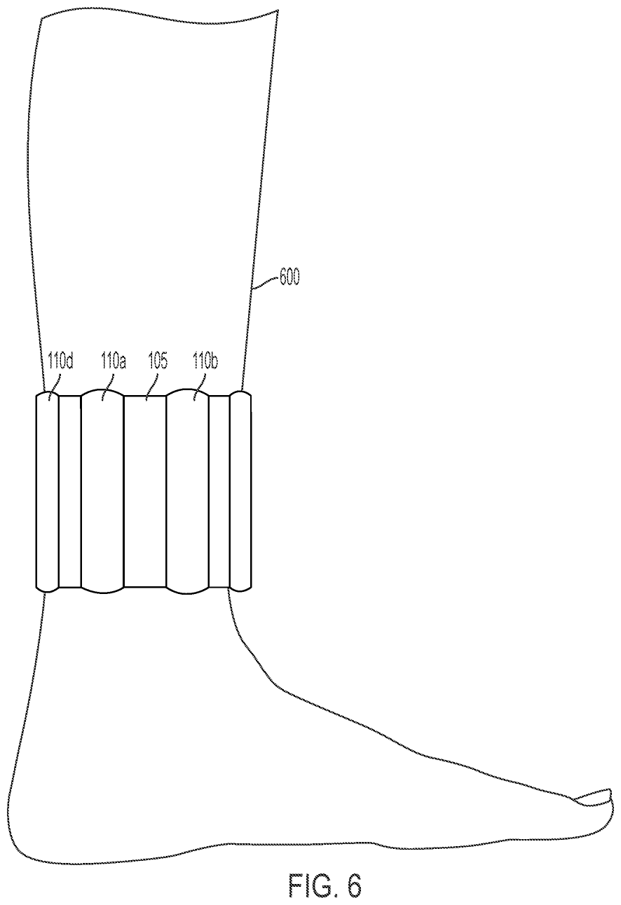

Referring to FIG. 6, depicted is the temperature control device 100 disposed around a body part 600. In some implementations, the body part 600 is a leg, arm, shoulder, torso, or a limb. In some implementations, the body part 600 is of a human or any other creature. For implementations in which cooling is desired, the temperature control device 100 may provide a cold compress for applying to a body part 600 that is injured. A pouch 110c may be folded on a pouch 110d to create a two-pouch cooling zone. The two-pouch cooling zone is secured by engaging a fastener 205b adjacent to the pouch 110c to a fastener complementary portion 210c adjacent to the pouch 110d. The temperature control device 100 attaches the two-pouch cooling zone to the body part 600. The two-pouch cooling zone targets the injury of the body part 600. In some implementations, the temperature control device 100 provides cooling to burn areas on a body part 600. In some implementations, the body part 600 includes a fastener 205 or fastener complementary portion 210. In some implementations, the temperature control device 100 is coupled to the body part 600. In other implementations, the temperature control device 100 is coupled anywhere on the body part 600. In some implementations, the temperature control device 100 is wrapped around the body part 600. In some implementations, the temperature control device 100 wraps anywhere on the body part 600. The temperature control device 100 is secured to the body part 600 by engaging a fastener 205 or a fastener complementary portion 210 of the temperature control device 100 to a fastener 205 or fastener complementary portion 210 of the temperature control device 100 to dispose the temperature control device 100 in a continuous, circular, or square shape.

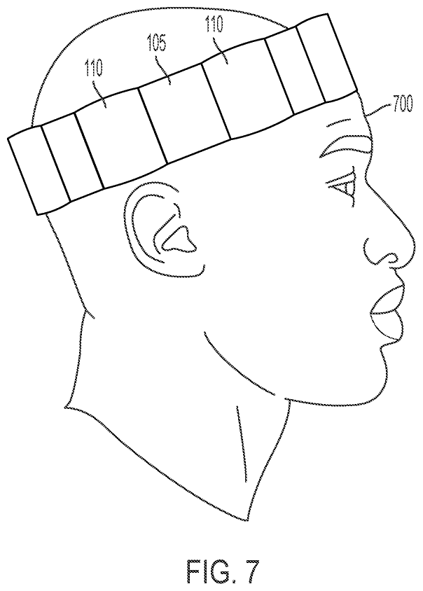

Referring to FIG. 7, depicted is the temperature control device 100 disposed around a head 700. In some implementations, the head 700 includes varying lengths of hair. In some implementations, the head 700 is of a human or any other creature. In some implementations, the temperature control device 100 is a cold compress for headaches. In some implementations, the temperature control device 100 wraps around the head 700. In some implementations, the temperature control device 100 is wrapped around the head 700. In some implementations, the temperature control device 100 wraps anywhere on the head 700. In some implementations, the temperature control device 100 secures to the head 700 by engaging a fastener 205 or a fastener complementary portion 210 of the temperature control device 100 to a fastener 205 or fastener complementary portion 210 of the temperature control device to dispose the temperature control device 100 in a continuous, circular, or square shape.

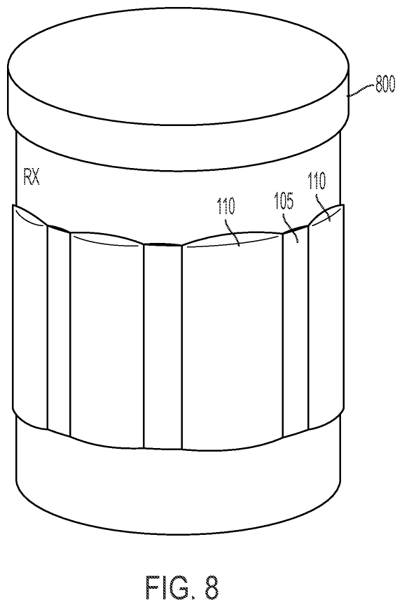

Referring to FIG. 8, depicted is the temperature control device 100 disposed around a medicine container 800. The medicine container 800 may or may not include a fastener 205 or fastener complementary portion 210. In some implementations the temperature control device 100 is coupled to the medicine container 800. In some implementations, the temperature control device 100 is coupled anywhere on the medicine container 800. In some implementations, the temperature control device 100 is wrapped around the medicine container 800. In some implementations, the temperature control device 100 wraps anywhere on the medicine container 800. The temperature control device 100 is secured to the medicine container 800 by engaging a fastener 205 or a fastener complementary portion 210 of the temperature control device 100 to a fastener 205 or fastener complementary portion 210 of the temperature control device or medicine container 800 to dispose the temperature control device 100 in a continuous, circular, or square shape.

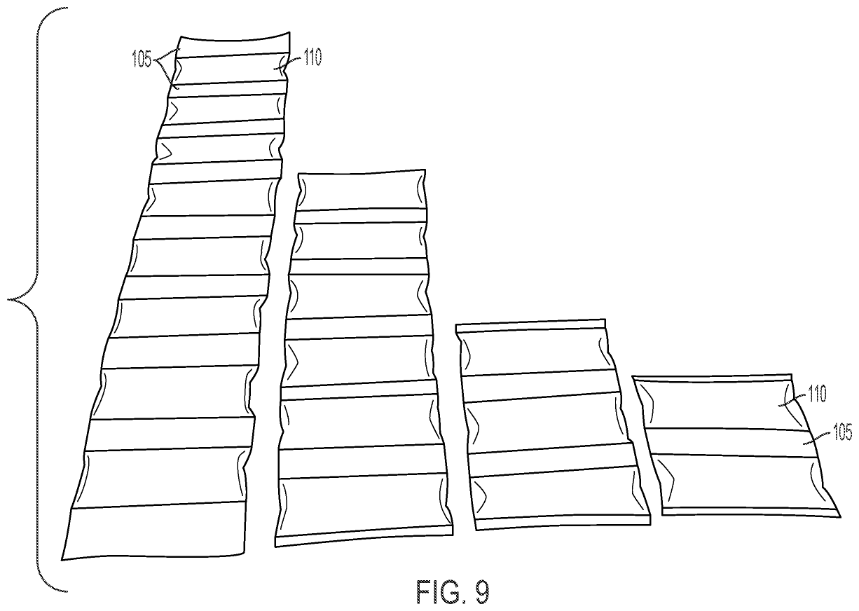

Referring to FIG. 9, depicted are several exemplary implementations of the temperature control device 100. In some implementations, each pouch 110 contains a different amount of temperature control substance. In some implementations, each flap 105 has either a fastener 205, a fastener complementary portion 210, or both. As shown in FIG. 9, the temperature control devices include an assortment of pouches 110. Temperature control occurs with the temperature control device 100, which comes in configurations of 2, 3, 4, 6, 8, 10, or any other custom number of pouches.

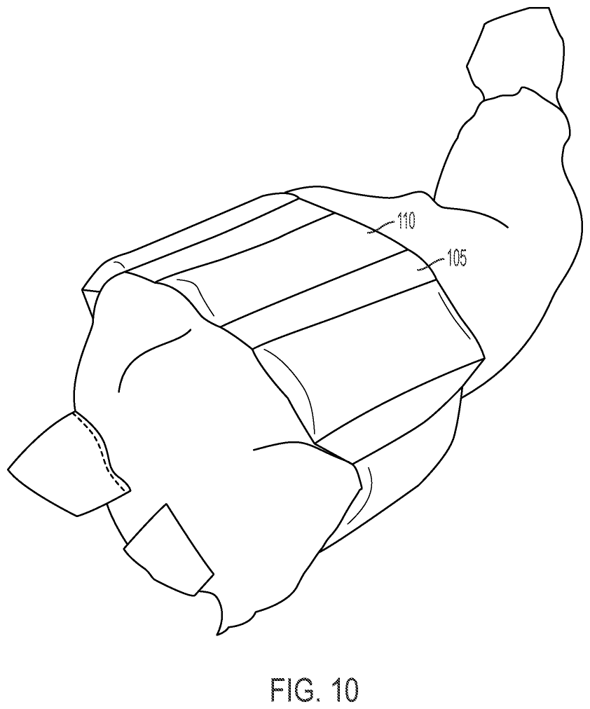

Referring to FIG. 10, depicted is an example implementation of the temperature control device 100 secured around an item. As shown in FIG. 10, the temperature control device 100 has been wrapped around an item to provide temperature control. The flap 105 includes the fastener 205 and the fastener complementary portion 210. The fastener 205 is affixed to the fastener complementary portion 210 to secure the temperature control device 100. The temperature control device 100 is secured to the item by affixing a fastener 205 to a fastener complementary portion 210. In some implementations, the temperature control device 100 attaches to an item by affixing a fastener 205 of the temperature control device 100 to a fastener complementary portion 210 of the item, or by affixing a fastener complementary portion 210 of the temperature control device 100 to a fastener 205 of the item.

Various elements, which are described herein in the context of one or more embodiments, may be provided separately or in any suitable subcombination. Further, the processes described herein are not limited to the specific embodiments described. For example, the processes described herein are not limited to the specific processing order described herein and, rather, process blocks may be re-ordered, combined, removed, or performed in parallel or in serial, as necessary, to achieve the results set forth herein.

It will be further understood that various changes in the details, materials, and arrangements of the parts that have been described and illustrated herein may be made by those skilled in the art without departing from the scope of the following claims.

We claim:

1. A modular temperature control system comprising:
a first flap portion and a second flap portion defining between them a first pouch containing a substance;
a third flap portion and the second flap portion defining between them a second pouch containing the substance;
a first portion of a fastener attached to the front side of the first flap portion;
a first fastener complementary portion attached to an obverse side of the second flap portion; and
a second fastener complementary portion attached to the obverse side of the third flap portion.

2. The modular temperature control system of claim 1, further comprising:
a second portion of the fastener attached to the front side of the second flap portion.

3. The modular temperature control system of claim 2, wherein the first portion of the fastener has a first surface area equal to a second surface area of the second portion of the fastener.

4. The modular temperature control system of claim 2, wherein the first portion of the fastener is a square shape.

5. The modular temperature control system of claim 1, wherein the first fastener complementary portion has a third surface area equal to a fourth surface area of the second fastener complementary portion.

6. The modular temperature control system of claim 1, wherein the first fastener complementary portion is a square shape.

7. The modular temperature control system of claim 1, wherein the substance is a gel.

8. The modular temperature control system of claim 1, wherein the substance is propylene glycol and water.

9. The modular temperature control system of claim 1, wherein the first pouch has a different amount of the substance from the second pouch.

10. The modular temperature control system of claim 1, wherein a surface area of the first, second, and third flap and a surface area of the first pouch and the second pouch are equal.

11. The modular temperature control system of claim 1, wherein the first fastener complementary portion comprises a strip of material.

12. The modular temperature control system of claim 1, wherein the first portion of the fastener comprises a strip of material.

13. The modular temperature control system of claim 1, wherein the second fastener complementary portion comprises a strip of material.

14. The modular temperature control system of claim 2, wherein the second portion of the fastener comprises a strip of material.

15. The modular temperature control system of claim 1, the first fastener complementary portion a plurality of shapes attached to the obverse side of the first flap portion.

16. A method for applying targeted temperature control comprising:
providing a first temperature control apparatus that comprises:
a first flap portion and a second flap portion defining between them a first pouch containing a substance;
a third flap portion and the second flap portion defining between them a second pouch containing the substance;
a first portion of a fastener attached to the front side of the first flap portion;
a first fastener complementary portion attached to an obverse side of the second flap portion; and
a second fastener complementary portion attached to the obverse side of the third flap portion; and
wrapping the first temperature control apparatus around an item.

17. The method for applying targeted temperature control of claim 16, further comprising:
securing the first temperature control apparatus to the item by affixing the first portion of the fastener to the first fastener complementary portion.

18. The method for applying targeted temperature control of claim 16, further comprising:
securing the first temperature control apparatus to the item by affixing the first portion of the fastener to the second fastener complementary portion.

19. The method for applying targeted temperature control of claim 16, further comprising:
providing a second temperature control apparatus comprising:
a first flap portion and a second flap portion defining between them a first pouch containing the substance;
a third flap portion and the second flap portion defining between them a second pouch containing the substance;
a first portion of a fastener attached to the front side of the first flap portion;
a first fastener complementary portion attached to the obverse side of the second flap portion; and
a second fastener complementary portion attached to the obverse side of the third flap portion;
attaching the first portion of the second temperature control apparatus to the second fastener complementary portion of the first temperature control apparatus to form a combined apparatus;
wrapping the combined apparatus around an item; and
securing the combined apparatus to the item by affixing the first portion of the first temperature control apparatus to the second fastener complementary portion of the first temperature control apparatus.

20. The method for applying targeted temperature control of claim 19, further comprising:
coupling the first fastener complementary portion of the first temperature control apparatus and the first fastener complementary portion of the second temperature control apparatus to a cover comprising a first fastener complementary portion and a second fastener complementary portion.
